(19) United States
(12) Patent Application Publication
(10) Pub. No.: US 2025/0144259 A1
(43) Pub. Date: May 8, 2025

(54) SYSTEMS, APPARATUS AND METHODS FOR STERILIZING AN OBJECT USING A SELF-CONTAINED STERILIZATION CHAMBER

(71) Applicant: CSP Technologies, Inc., Auburn, AL (US)

(72) Inventors: Brian Tifft, Auburn, AL (US); Gary Anderson, Auburn, AL (US); John Belfance, Auburn, AL (US)

(21) Appl. No.: 18/837,945

(22) PCT Filed: Feb. 17, 2023
(86) PCT No.: PCT/US2023/062785

§ 371 Date: Aug. 13, 2024

Related U.S. Application Data
(60) Provisional application No. 63/268,164, filed on Feb. 17, 2022.

(51) Int. Cl. A61L 2/24; A61L 2/20; A61L 2/26; A61L 101/06; B01D 46/00; B01D 46/46

(52) U.S. Cl. CPC A61L 2/24; A61L 2/20; A61L 2/26; B01D 46/0036; B01D 46/46; A61L 2101/06; A61L 2202/11; A61L 2202/121; A61L 2202/122; A61L 2202/123; A61L 2202/13; A61L 2202/14; A61L 2202/24

(57) ABSTRACT

Disclosed are systems for providing safe, conveniently onsite, quick and effective ways to deliver antimicrobial and antiviral treatment to objects. Further disclosed is an isolation unit for sterilizing objects, the isolation having a lockable hatch that enables access, when unlocked, to a sterilization chamber within the isolation unit. The hatch remains locked until a sterilization operation is completed. Further, the hatch is prevented from being opened until the sterilization operation is completed. This feature prevents potentially harmful sterilizing chemicals from emitting out of the sterilization chamber, among other possible benefits.

### CROSS-REFERENCE TO RELATED APPLICATIONS

This application claims priority to U.S. Provisional Patent Application No. 63/268,164, entitled “SYSTEMS, APPARATUS AND METHODS FOR STERILIZING AN OBJECT USING A SELF-CONTAINED STERILIZATION CHAMBER,” filed on Feb. 17, 2022, the contents of which are incorporated by reference herein in their entirety.

### FIELD OF THE INVENTION

This invention relates to systems, apparatus and methods for disinfection or sterilization of objects, specifically by use of polymer compositions having antimicrobial properties that release sterilizing gas, e.g., chlorine dioxide gas. Chlorine dioxide gas functions in accordance with the invention as an antimicrobial agent to inhibit pathogens. Further, the system herein provides a self-contained chamber to perform sterilizing operations using the polymer composition. The system herein will have application in the reduction, inhibition and elimination of viral, bacterial, fungal and other microbial proliferation or infection. Of particular use, the system herein may be used for the disinfection or sterilization of medical devices, including protective personal equipment, medical equipment, surgical instruments and other reusable objects that require sterilization in a medical setting before use on or in a patient or medical professional. In this way, the devices may be disinfected or sterilized and reused multiple times, enhancing safety to healthcare professionals and patients and addressing limited supply of such items. The disinfection or sterilization of any other myriad of objects is also contemplated, including cell phones, cosmetics, kitchenware, toys, eyeglasses, mail, currency and others.

### BACKGROUND OF THE INVENTION

Medical professionals must ensure that reusable medical equipment and devices (e.g., surgical instruments and endoscopes) are free of pathogens before using such objects on themselves (e.g., such as wearing N95 masks), or on or in patients. Various methods exist to sterilize and thus reprocess medical equipment and devices, including ionizing radiation, sterilization with ethylene oxide (EtO), microwave-generated steam (MGS), ultraviolet germicidal irradiation (UVGI), gamma radiation, steam, bleach, liquid hydrogen peroxide (LHP), and hydrogen peroxide gas plasma (HPGP). However, these decontamination procedures either require specialized materials, equipment, or facilities, and may be unsafe unless properly performed by a professional with specialized training.

The Battelle Critical Care Decontamination System™ (from Battelle Memorial Institute of Columbus, Ohio, USA) received FDA Emergency Use Authorization and became available in March 2020 in response to the Covid-19 pandemic. The Battelle decontamination system purported successful testing on decontaminated N95 respirators demonstrating acceptable performance through 20 decontamination cycles for sporicidal activity, viricidal activity, filtration efficiency, breathability, form fit testing, and strap integrity testing, per authorized respirator. The Battelle system is a self-contained decontamination device that uses vapor phase hydrogen peroxide (VPHP) for decontamination of compatible N95 or N95-equivalent respirators that are contaminated or potentially contaminated with SARS-CoV-2. Each decontamination cycle in the Battelle decontamination system consists of injecting VPHP into the decontamination chamber until achieving a saturated atmosphere indicated by micro condensation; maintaining the VPHP exposure for a 150-minute dwell time; and allowing the VPHP to off gas to a level of 1 ppm prior to post decontamination processing. A minimum of five calibrated chemical indicators are dispersed throughout the system to indicate a successful decontamination cycle. One drawback of the Battelle system is that it requires complicated and expensive specialized equipment to operate. For example, the Battelle system requires a complicated means to generate the VPHP from outside the sterilization chamber and means to deliver the gas into the chamber to effectuate sterilization.

Chlorine dioxide (ClO₂) has been shown to be effective as an antimicrobial agent in reducing pathogens. It has also been shown to be effective against a variety of viruses. Products containing ClO₂ gas are used for agricultural, commercial, industrial, medical and residential use antibacterial application. Specifically, the gaseous effects of chlorine dioxide against Influenza A were studied by Ogata, Samp and Shibata in 2008. This team showed that 0.03 ppm of ClO₂ could have an effective 4 log kill when administered at the same time as the virus and if administered after the survival rate increased to 100% versus 30% when untreated. Ogata, N., Shibata, T. Protective effect of low-concentration chlorine dioxide gas against influenza A virus infection. Journal of General Virology, 89(1), 60-67, (2008). Harakeh gives data that shows certain types of viruses can be inactivated by as much as or more than 99.9% by 4 ppm concentration of ClO₂ after 5 minutes of exposure, including human rotavirus, coxsackievirus B5, echovirus 1, poliovirus 1, bacteriophage f2, and siamian rotovirus. Harakeh, S. The behavior of viruses on disinfection by chlorine dioxide and other disinfectants in effluent. FEMS Microbiology Letters, 44(3), 335-341, (1987). A study by Sanekata et al showed that a concentration of 1.0 ppm of ClO₂ for 180 seconds could achieve a 2 to 4 log kill on Infectious Flacherie Virus (IFV), measles, and HHV-1. Sanekata, T., Fukuda, T., Miura, T., Morino, H., Lee, C., Maeda, K., Shibata, T. Evaluation of the Antiviral Activity of Chlorine Dioxide and Sodium Hypochlorite against Feline Calicivirus, Human Influenza Virus, Measles Virus, Canine Distemper Virus, Human Herpesvirus, Human Adenovirus, Canine Adenovirus and Canine Parvovirus. Biocontrol Science, 15(2), 45-49, (2010). Simonet, Samp, and Gantzer showed that polio can also be inactivated with exposure to chlorine dioxide. Simonet, J., Gantzer, C. Degradation of the Poliovirus 1 genome by chlorine dioxide. Journal of Applied Microbiology, 100(4), 862-870, (2006).

In light of its demonstrated safety and efficacy, ClO₂ gas sterilization shows much promise as an alternative to other gas sterilization methods. However, as with other forms of gas sterilization, conventional equipment for generating and feeding ClO₂ gas into a sterilization chamber is complicated and expensive. In view of this problem, Applicant had developed a new technology to simply, safely and effectively generate ClO₂ gas. This technology is described in International Patent Application No. PCT/US2019/060937 and in U.S. Publication No. 2019/0335746 A1. Disclosed in PCT/US2019/060937 is a chlorine dioxide gas forming agent that is provided with a carrier material within a polymer composition that comprises silica gel which is preferably acidified. Optionally, the chlorine dioxide gas forming agent comprises a carrier material (e.g., silica gel), an active compound (metal chlorite, such as sodium chlorite) and a moisture trigger (hygroscopic compound, e.g., calcium chloride). The carrier material preferably comprises an acidified silica gel having a pH of from 1.4 to 3.1 and is 50% to 90% by weight with respect to the total weight of the antimicrobial releasing agent. The active compound is from 5% to 30% by weight with respect to the total weight of the antimicrobial releasing agent. The trigger is from 2% to 20% by weight with respect to the total weight of the antimicrobial releasing agent. In one optional embodiment, the chlorine dioxide gas forming agent comprises, from 10% to 15% sodium chlorite, from 5% to 15% calcium chloride, and from 70% to 80% silica gel by weight based on the total weight of the chlorine dioxide gas forming agent. Preferably, the carrier of the chlorine dioxide gas forming agent has a pH of from 1.0 to 3.5, optionally from 1.4 to 3.1. Preferably, the chlorine dioxide gas forming agent is a component of a polymer composition that comprises a base polymer, a channeling agent and the chlorine dioxide gas forming agent.

The aforementioned technology, particularly in the form of the polymer composition, provides a significant advance in that it may be configured to generate predetermined amounts of ClO₂ gas with precision and within predetermined timeframes. Moreover, it may be embodied in a polymer composition—what ostensibly appears to be a simple piece of plastic—that when exposed to moisture, is triggered to generate chlorine dioxide gas. The polymer composition does not require any specialized equipment, electronics, pumps, etc., to generate ClO₂ gas. It is thus readily portable, simple and comparatively inexpensive to use.

Challenges to any gas sterilization process include ensuring that: (1) the gas is substantially contained so as not to permeate into the ambient environment; and (2) the process cycle is completed so as to effectuate the necessary level of sterilization. In the context of ClO₂ gas sterilization using Applicant's polymer composition technology, there is a need to provide a chamber that prevents escape of the gas during the cycle, that ensures that the ClO2 gas is gone or present below acceptable levels within the chamber after the cycle and that prevents inadvertent or deliberate interruptions to the cycle or premature removal of the object(s) subjected to sterilization.

An additional need exists for a sterilization system for medical devices and equipment that is uncomplicated to use, with simple procedure of use and simple instructions, so that any healthcare worker, as well as an average person without healthcare or scientific training, can easily comprehend and learn to utilize the system.

### SUMMARY OF THE INVENTION

Accordingly, in one aspect, disclosed herein is a system for sterilization or disinfection, which includes a self-contained sterilization chamber that uses a fail-deadly locking mechanism that remains locked once triggered and can only be unlocked when certain conditions are satisfied. The system can be easily and quickly deployed across the world and is readily scalable. The system herein is useful for the disinfection, decontamination, sanitation and/or sterilization of any object in general and peculiarly medical devices and medical equipment. The system preferably includes the use of polymer compositions incorporating a chlorine dioxide gas forming agent which is capable of forming and releasing chlorine dioxide gas as an active agent that functions to inhibit microbial proliferation. The system is contemplated for use in hospital settings and is especially well-suited for smaller clinics, dental offices, urgent care centers, nursing homes, university clinics, military field hospitals and tribal and rural healthcare facilities. In total, it provides a very practical means of satisfying government reprocessing guidelines to safely and effectively sterilize medical devices or equipment for reuse. A particular benefit of the disclosed system is that it is scalable to as many treatment units as are necessary for a desired application.

The system may comprise an isolation unit, at least one hatch, at least one locking mechanism, at least one human machine interface (HMI) device, at least one output device, and a control system. Further, the hatch may be laterally integrated into the isolation unit. Further, the locking mechanism may be operatively coupled in between the isolation unit and the hatch, wherein the locking mechanism functions as a fail-deadly switch to prevent opening the hatch once the locking mechanism is engaged. Further, the HMI device may be laterally mounted onto the isolation unit. Further, the output device may be laterally mounted onto the isolation unit. Further, the control system may be disposed within the isolation unit. Further, the control system may be communicably coupled to the HMI device and the output device.

Further, the isolation unit may comprise a sterilization chamber and a component enclosure. Further, the hatch may be integrated into the sterilization chamber, wherein an interior compartment of the sterilization chamber is selectively accessible through the hatch. Further, the component enclosure may be laterally mounted onto the sterilization chamber and positioned offset from the hatch. Further, the HMI may comprise at least one control button and at least one keylock switch. Further, the keylock switch and the control button may be laterally mounted onto the component enclosure. Further the output device may comprise at least one status light, at least primary display device, and at least secondary display device.

Further, the hatch may comprise an opening and a door. Further, the opening may traverse through the sterilization chamber into the interior compartment. Further, the door may be mounted over the opening. Further, the locking mechanism may be connected in between the door and the sterilization chamber.

Further, the locking device may comprise an engagement device and a receptacle. Further, the engagement device may be mounted onto the hatch. Further, the receptacle may be mounted onto the sterilization chamber. Further, the engagement device may be selectively engaged into the receptacle, wherein this engagement prevents the opening from being accessed through the door. Further, the engagement device may comprise at least one locking prong and at least one biasing device. Further, the locking prong may be operatively coupled to the biasing device, wherein the biasing device forces the locking prong to selectively engage into the receptacle. Further, the engagement device may comprise at least one reset lever. Further, the reset lever may be operatively coupled to the biasing device, wherein the reset lever forces the locking prong to disengage from the receptacle.

Further, the disclosed concept may comprise an environmental analysis instrumentation suite. Further, the environmental analysis instrumentation suite may be mounted within the interior compartment of the sterilization chamber. Further, the control system may be communicably coupled to the locking mechanism and the environmental analysis instrumentation suite.

Further, the disclosed concept may comprise a rack assembly, an element tray, and a sterilizing element. Further, the rack assembly may be mounted within the interior compartment of the sterilization chamber. Further, the element tray may be mounted within the interior compartment. Further, the sterilizing element may be positioned on the element tray.

In one particular embodiment, disclosed is a system that uses sterilizing elements that are antimicrobial strips formed using three-phase entrained polymer technology (Activ-Shield™ technology by Aptar CSP Technologies Inc., Auburn AL, USA.) In alternate embodiments, the chloride dioxide gas forming agents used herein include chlorite salts, including alkali metal chlorites, alkaline earth metal chlorite or a transition metal chlorite. Moisture activates the metal chlorite to form chlorine dioxide gas.

The three-phase polymer provides the ability to control small molecule transport through the polymer. The pathways created by the interaction of these constituents allow for the controlled movement of chlorine dioxide gas into and out of the polymer. This has enabled the ability to engineer compounds that transmit ClO₂ gas molecules. When released into a sealed atmosphere of a package, the entrained polymer allows maintaining an optimal environment with minimal or reduced microbial count.

One method according to an optional aspect of the invention comprises the following steps: (a) placing the object to be disinfected into an isolation unit having an interior space therein, a headspace being formed of a portion of the interior space that is not occupied by the object; (b) placing into the interior space a polymer composition comprising: (i) a base polymer; (ii) a chlorine dioxide gas forming agent; and (iii) a channeling agent that forms channels though the base polymer; (c) contacting the polymer composition with moisture to form chlorine dioxide gas; and (d) enclosing the isolation unit sufficiently enough to allow the chlorine dioxide gas to accumulate in the headspace, wherein the chlorine dioxide gas disinfects the object; wherein the amount of chlorine dioxide gas on the disinfected object is substantially or completely undetectable immediately after removal of the object from the isolation unit; optionally, within 1 minute after removal; optionally within 5 minutes after removal; optionally within 10 minutes after removal; or optionally within one hour after removal; alternatively, wherein the amount of chlorine dioxide gas on the disinfected object is less than 0.01 ppm immediately after removal of the object from the isolation unit; optionally, within 1 minute after removal; optionally within 5 minutes after removal; optionally within 10 minutes after removal; alternatively, wherein the amount of chlorine dioxide gas in the ambient environment around the isolation unit is substantially or completely undetectable the entire time that the method is performed and optionally immediately after removal of the object from the isolation unit, optionally within 1 minute after removal; alternatively, wherein the amount of chlorine dioxide gas in the ambient environment around the isolation unit is less than 0.01 ppm the entire time that the method is performed and optionally immediately after removal of the object from the isolation unit, optionally, within 1 minute after removal; alternatively, wherein the amount of chlorine dioxide gas in the ambient environment around the isolation unit is considered Generally Recognized as Safe (GRAS) pursuant to Sections 201(s) and 409 of the United States Federal Food, Drug, and Cosmetic Act the entire time that the method is performed and optionally immediately after removal of the object from the isolation unit, optionally, within 1 minute after removal.

The amount of chlorine dioxide formed by the polymer composition is controlled by several means. In one optional embodiment, the amount of chlorine dioxide gas released into a room is 0.001 ppm to 0.1 ppm over an average of a 10-hour work shift. In an alternate optional embodiment, the amount of chlorine dioxide released is approximately 0.3 ppm over a time period of 15 minutes. Such embodiments are the ranges considered as safe for use by humans by the U.S. Center of Disease Control and Prevention (CDC), but are not limited thereto.

Optionally, in any embodiment involving disinfection of objects contaminated by microbes, the concentration of chlorine dioxide gas formed in the isolation unit effectuates a reduction of infectious viral or bacterial pathogens on the object to be disinfected, the reduction being at least a 1 log based 10 reduction in the number of such particles, optionally at least a 2 log based 10 reduction in the number of such particles, optionally at least a 3 log based 10 reduction in the number of such particles, optionally at least a 4 log based 10 reduction in the number of such particles, optionally at least a 5 log based 10 reduction in the number of such particles, optionally at least a 6 log based 10 reduction in the number of such particles, optionally at least a 7 log based 10 reduction in the number of such particles, optionally at least a 8 log based 10 reduction in the number of such particles, as compared to the initial number of such particles.

In accordance with another aspect of the disclosed concept, the profile release rate and duration of chlorine dioxide gas formation can be designed and controlled.

It is yet an additional important component of embodiments of the invention that during the disinfection process, the amount of chlorine dioxide gas in the ambient environment around the isolation unit is substantially or completely undetectable the entire time that the method is performed and optionally immediately after removal of the object from the isolation unit, optionally within 1 minute after removal.

Optionally, colorants or color indicators are added to the polymer composition as standalone indicators to indicate chlorine dioxide activity. Optionally, the concentration of a colorant is approximately 1% to 3%, optionally about 2% of the total weight of the polymer composition. A marker or similar gage can also be used to indicate and monitor the activity of the chlorine dioxide gas in the system.

### BRIEF DESCRIPTION OF THE DRAWINGS

The invention will be described in conjunction with the following drawings in which like reference numerals designate like elements and wherein:

FIG. 1 is a front view of an isolation unit that may be used in the disclosed concept.

FIG. 2 is a right-side view of the isolation unit of FIG. 1.

FIG. 3 is a top view of the isolation unit of FIG. 1.

FIG. 4 is a block diagram illustrating the arrangement of components within the interior compartment of the isolation unit according to an optional aspect of the disclosed concept.

FIG. 5 is a block diagram illustrating a temperature control system and a humidity control system disposed within the interior compartment of the isolation unit according to an optional aspect of the disclosed concept.

FIG. 6 is a block diagram illustrating an air purification system disposed within the interior compartment of the isolation unit according to an optional aspect of the disclosed concept. Here, dashed lines indicate the direction of airflow through the air purification system.

FIG. 7 is a block diagram illustrating communicably coupled components of the isolation unit according to an optional aspect of the disclosed concept.

FIG. 8 is a flowchart illustrating a method for sterilizing an object using the isolation unit according to an optional aspect of the disclosed concept.

FIG. 9 is a flowchart illustrating a method for sterilizing an object using the isolation unit according to an aspect of the disclosed concept.

### DETAILED DESCRIPTION OF THE INVENTION

### Definitions

As used herein, the term “antimicrobial” or “antimicrobial agent” refers to a substance that inhibits microorganisms. Classes of antimicrobials include antivirals, antibacterials, antifungals, antiparasites and other anti-pathogenic agents.

As used herein, the term “base polymer” is a polymer that is capable of being formed with a chlorine dioxide gas forming agent, and optionally having a gas transmission rate of a selected material that is substantially lower than, lower than or substantially equivalent to, that of a channeling agent mixed into the base polymer. By way of example, such a transmission rate is a water vapor transmission rate in embodiments where the chlorine dioxide gas forming agent is activated by moisture. The primary function of the base polymer is to provide structure for with a polymer composition comprising the base polymer, the chlorine dioxide gas forming agent and preferably a channeling agent.

As used herein, the term “channeling agent” is defined as a material that is immiscible with the base polymer and has an affinity to transport a gas phase substance at a faster rate than the base polymer alone. Optionally, a channeling agent is capable of forming channels through the entrained polymer when formed by mixing the channeling agent with the base polymer. Optionally, such channels are capable of transmitting a selected material, such as water, chlorine dioxide or others, through the entrained polymer at a faster rate than then the selected material would have in the base polymer without the channeling agent. As used herein, the term “channels” or “interconnecting channels” is defined as passages formed of the channeling agent that penetrate through the base polymer and may be interconnected with each other.

As used herein, the term “chlorine dioxide gas forming agent” refers to a compound that upon contact with moisture, or potentially in response to another trigger, reacts to form chlorine dioxide, which is released in gas form.

As used herein, the terms “close”, “closed” and “closing” are used interchangeably with the terms “seal”, “sealed” and “sealing”, respectively, to refer to an isolation chamber of an isolation unit an being enclosed sufficiently enough to the extent that the amount of chlorine dioxide gas that is formed and remains inside the isolation unit, when enclosed is on balance greater than the amount of chlorine dioxide gas that exits the isolation unit, such that the chlorine dioxide gas accumulates inside the isolation unit and reaches a measurable concentration isolation unit therein. Sealing is needed to minimize permeation of both moisture and chlorine dioxide gas through the isolation unit wall and ingress through the seal, which will also be a factor of the amount of time that the object is retained in the isolation unit to dwell in the chlorine dioxide gas and thereby become sterilized or disinfected isolation unit.

As used herein, the terms “decontamination”, “disinfection”, “sanitization”, and “sterilization” (and “decontaminate”, “disinfect”, “sanitize”, and “sterilize” and conjugated forms thereof,) are defined herein to mean the action of contacting an object with chlorine dioxide in order to inhibit an infectious agent such as a bacteria, a virus, a fungus, a parasite or other.

The terms decontamination, disinfection, sanitization, and sterilization are often colloquially used interchangeably in general lexicon. Varying definitions are available and are provided by way of background, information and guidance in interpretation, but the terms are used as defined herein. The terms have also acquired certain meanings within various fields of practice, (e.g., in chemistry, medicine, food science, and others). These terms are also are specially defined by various organizations for specific purposes, such as the U.S. Center for Disease Control (CDC), Environmental Protection Agency (EPA) and Food and Drug Administration (FDA). The definitions set forth by the CDC, EPA and FDA are provided by way of example and information only and are not intended to be limiting, unless otherwise stated in a given instance or claim. Furthermore, for purposes of clarity, the terms “decontamination”, “disinfection”, “sanitization”, “sterilization” (as well as the terms “decontaminate”, “disinfect”, “sanitize”, and “sterilize” and their conjugated forms) are used interchangeably with one another herein and each time one of these terms is used throughout the specification and claims, such term is intended to include and cover all four terms and iterations listed, unless otherwise indicated in a given instance or claim.

The U.S. CDC provides the following definitions: “Decontamination: The use of physical or chemical means to remove, inactivate, or destroy blood borne pathogens on a surface or item to the point where they are no longer capable of transmitting infectious particles and the surface or item is rendered safe for handling, use, or disposal. In health-care facilities, the term generally refers to all pathogenic organisms.” “Disinfection: Thermal or chemical destruction of pathogenic and other types of microorganisms. Disinfection is less lethal to microbes than sterilization because it destroys most recognized pathogenic microorganisms but not necessarily all microbial forms (e.g., bacterial spores).” “Sanitizer: Agent that reduces the number of bacterial contaminants to safe levels as judged by public health requirements. The term is commonly used with substances applied to inanimate objects. According to the protocol for the official sanitizer test, a sanitizer is a chemical that kills 99.999% of the specific test bacteria in 30 seconds under the conditions of the test.” “Sterile or Sterility: State of being free from all living microorganisms. In practice, these terms are usually described as a probability function, e.g., as the probability of a microorganism surviving sterilization being one in one million.” “Sterilization: Validated process used to render a product free of all forms of viable microorganisms. In a sterilization process, the presence of microorganisms on any individual item can be expressed in terms of probability. Although this probability can be reduced to a very low number, it can never be reduced to zero.” [C.F.R. Title 29 Section 1910.1030] (see https://www.cdc.gov/infectioncontrol/guidelines/disinfection/glossary.html). As used herein, the term “sterilized” in addition to and consistent with the definition above, (meaning an object contacted with chlorine dioxide for sufficient concentration and time in order to inhibit a microorganism), includes that the object is free of all forms of viable microorganisms, described as a probability of a microorganism surviving sterilization being one in one million, as set forth by the U.S. CDC pursuant to the Code of Federal Regulations, Title 21, Section 110.3(o).

The U.S. EPA defines “sanitizer” as “a substance, or mixture of substances, that reduces the bacteria population in the inanimate environment by significant numbers, but does not destroy or eliminate all bacteria.” The term “disinfectant” is defined as “a substance or mixture of substances, that destroys or irreversibly inactivates bacteria, fungi, and viruses, but not necessarily bacterial spores, in the inanimate environment.” [C.F.R. Title 40, Section 158.2203.]

The U.S. FDA defines “sterilization,” in a document titled “Liquid Chemical Sterilants/High Level Disinfectants Guidance”, (https://www.cdc.gov/infectioncontrol/guidelines/disinfection/tables/table1.html), as “a validated process used to render a product free of all forms of viable microorganisms. In many cases, thermal methods, such as steam, are used to achieve sterilization. Thermal sterilization methods have been studied and characterized extensively. In addition, the survival kinetics for gas/vapor/plasma low temperature sterilization methods have also been well characterized.” “Sanitize” is defined as: “means to adequately treat food-contact surfaces by a process that is effective in destroying vegetative cells of microorganisms of public health significance, and in substantially reducing numbers of other undesirable microorganisms, but without adversely affecting the product or its safety for the consumer.” [C.F.R. Title 21, Section 110.3(o).]

As used herein, the term “headspace” refers to the portion of the interior space of an isolation unit that is not occupied by an object within the isolation unit.

As used herein, the term “infectious agent” refers to a microorganism of any species of a virus, bacteria, fungus, algae, parasite, other microbe that is capable of infecting a living organism and is capable of being modified by contact with chlorine dioxide. The infectious agent is typically but not necessarily a pathogen. The terms “infectious agent”, “microbial agent” and “pathogen” are used interchangeably herein.

As used herein, the term “inhibit” refers to the ability of chlorine dioxide to modify, hinder, restrain, prohibit, reduce, halt, inactivate, kill, stop or essentially prevent an infectious agent in its capacity to grow and/or proliferate and/or to infect another organism. All such terms are used interchangeably herein. The inhibition of antimicrobial growth may further aid in the prevention of infectious diseases caused by the virus, bacteria, fungus, algae, parasite or other microbial agents that are spread by persons touching an infected object, by airborne pathogenic transmission or by other mechanisms of transmission.

As used herein, the term “moisture” refers to and includes water (having the general chemical formula H₂O), steam (water in the form of steam), water vapor (water in its gaseous state, also commonly referred to as steam, which is typically vaporized by boiling or evaporation of water), vapor of liquid substances containing water, ambient air (ambient moisture in the environment containing water molecules or water in gas form), water molecules in a liquid other than water; as well as acetone, and/or alcohols including methanol, ethanol, propanol, butanol or ethylene glycol, as well as polar solvents, and/or combinations of any of the foregoing.

As used herein, the term “monolithic,” in “monolithic composition” is defined as a substance that is made of one essentially admixed or blended composition of materials, such that it does not itself consist of two or more discrete macroscopic layers or portions. Accordingly, a monolithic composition does not include a multi-layer composite, although a monolithic composition could form a layer of such a composite.

As used herein, the term “phase” is defined as a portion or component of a monolithic composition that is uniformly distributed throughout, to give the structure or composition its monolithic characteristics.

As used herein, the term “polymer composition” is defined as a monolithic material formed of at least a base polymer with the chlorine dioxide gas forming agent and optionally also a channeling agent distributed throughout the base polymer. A polymer composition thus includes two-phase polymers (without a channeling agent) and three phase polymers (with a channeling agent).

As used herein, the term “three phase” is defined as a monolithic composition or structure comprising three or more phases. An example of a three phase composition according to an optional aspect of the invention is an entrained polymer formed of a base polymer, chlorine dioxide gas forming agent, and channeling agent in an amount sufficient to form channels. Optionally, a three phase composition or structure may include none or more additional compounds, (e.g., a colorant), but is nonetheless still considered “three phase” on account of the presence of the three primary functional components.

As used herein, the term “N95” mask is defined as a respirator mask that satisfies the definition of an N95 mask pursuant to regulations of the National Institute for Occupational Safety and Health of the United States (NIOSH).

As used herein, the term “communicably coupled” shall mean that two or more electrical components are connected in such a way that power, information, or both may be exchanged between the coupled components.

### Polymer Compositions

The chlorine dioxide gas forming agent is a component of a polymer composition, preferably a three phase entrained polymer comprising the chlorine dioxide gas forming agent, a base polymer and a channeling agent. The polymer compositions herein are three phase formulations (i.e., comprising a base polymer, active agent and channeling agent). Entrained polymer compositions that include actives other than chlorine dioxide releasing agents (such as desiccants) are described, for example, in U.S. Pat. Nos. 5,911,937, 6,080,350, 6,124,006, 6,130,263, 6,194,079, 6,214,255, 6,486,231, 7,005,459, and U.S. Patent Publication No. 2016/0039955, each of which is incorporated herein by reference as if fully set forth. Entrained polymer compositions that include chlorine dioxide releasing agents are described in International Patent Application No. PCT/US2019/060937 and in U.S. Publication No. 2019/00335746 A1,which are each incorporated herein by reference in their entireties as if fully set forth herein.

Suitable base polymers include thermoplastic polymers, including but not limited to polypropylene, polyethylene, polyisoprene, polyhydroxyalkanoates (PHAs), polylactique acid (PLA), polybutylene succinate (PBS), polyhexene, polybutadiene, polybutene, polysiloxane, polycarbonate, polyamide, ethyl vinyl acetate, ethylene-vinyl acetate (EVA) copolymer, ethylene-methacrylate copolymer, polyvinyl chloride (PVC), polystyrene, polyester, polyanhydride, polyacrylianitrile, polysulfone, polyacrylic ester, acrylic, polyurethane, polyacetal, polyvinylpyrrolidone (PVP), a copolymer, and combinations thereof.

Optionally, in any embodiment, the concentration of the base polymer within the polymer composition is in a range from 10% to 80%, optionally from 20% to 70%, optionally from 30% to 60%, optionally from 40% to 50%, optionally from 45% to 65%, optionally from 45% to 60%, optionally from 45% to 55%, optionally from 50% to 70%, optionally from 50% to 60%, optionally from 55% to 65%, optionally from 55% to 60% by weight of the total weight of the polymer composition.

The polymer compositions herein incorporate channeling agents which preferably form channels between the surface of the polymer composition and its interior in order to transmit moisture or gas, to absorb or adsorb the moisture or gas, and to allow reaction of the moisture or gas with the chlorine dioxide gas forming agent. The channels are mainly formed of the channeling agent itself. The channeling agent used herein has a water vapor transmission rate of at least two times that of the base polymer. In other embodiments, the channeling agent has a water vapor transmission rate of at least five times that of the base polymer. In other embodiments, the channeling agent has a water vapor transmission rate of at least ten times that of the base polymer. In still other embodiments, the channeling agent may have a water vapor transmission rate of at least twenty, fifty or one hundred times that of the base polymer.

Suitable channeling agents include a polyglycol such as polyethylene glycol (PEG), ethylene-vinyl alcohol (EVOH), polyvinyl alcohol (PVOH), glycerin polyamine, polyurethane and polycarboxylic acid including polyacrylic acid or polymethacrylic acid. Alternatively, the channeling agent can be, for example, a water insoluble polymer, such as a propylene oxide polymerisate-monobutyl ether, such as polyglykol B01/240, produced by Clariant Specialty Chemicals. In other embodiments, the channeling agent could be a propylene oxide polymerisate monobutyl ether, such as polyglykol B01/20, produced by Clariant Specialty Chemicals, propylene oxide polymerisate, such as polyglykol D01/240, produced by Clariant Specialty Chemicals, ethylene vinyl acetate, nylon 6, nylon 66, or any combination of the foregoing.

Optionally, in any embodiment, the concentration of the channeling agent in the polymer composition is in a range from 1% to 25%, optionally from 2% to 15%, optionally from 5% to 20%, optionally from 8% to 15%, optionally from 10% to 20%, optionally from 10% to 15%, optionally from 10% to 12%, optionally from 5% to 15%, optionally about 7% by weight of the total weight of the polymer composition.

### Isolation Unit and Systems and Methods Using the Same

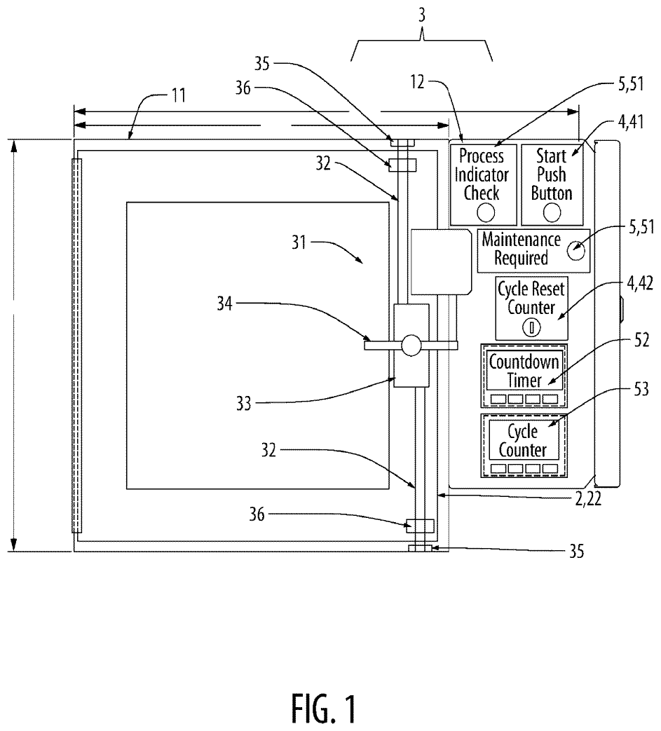

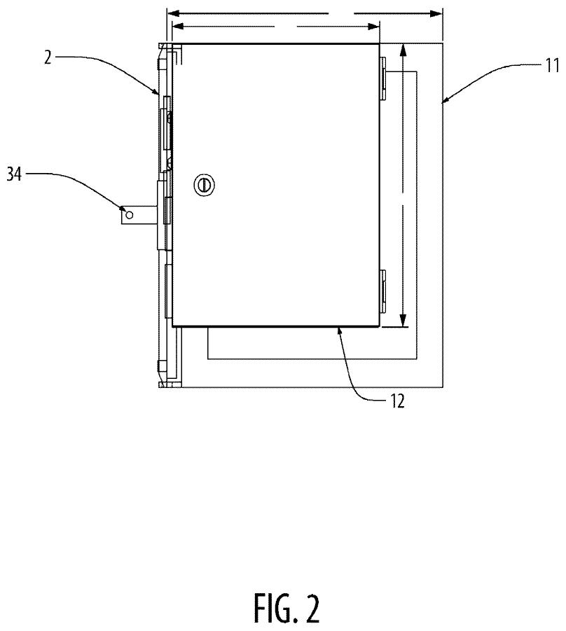

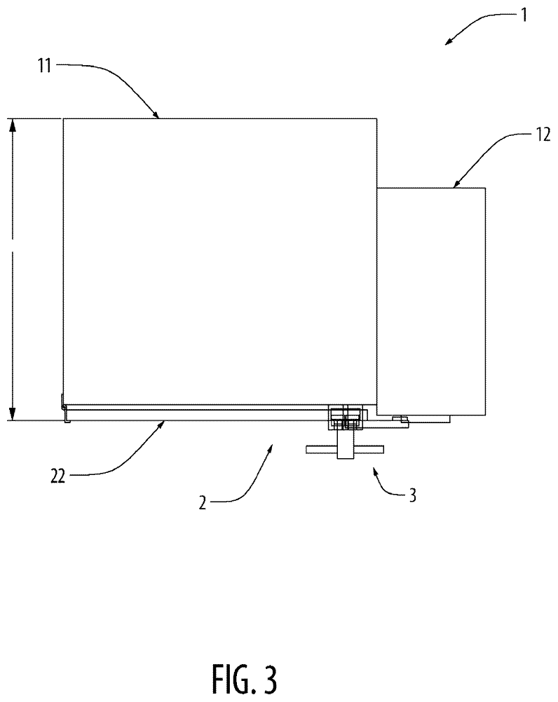

Referring generally to FIG. 1 through FIG. 9, the disclosed concept, in one aspect, is a system for sterilizing an object using a self-contained sterilization chamber of an isolation unit. Specifically, the system according to an aspect of the disclosed concept uses a sterilizing element that is positioned within the sterilization chamber thus making the chamber self-contained to release a quantity of sterilizing agent, preferably in gas form, into the self-contained sterilization chamber. This enables the system to sterilize the object without the need for external power supplies or fluid delivery systems for effectuating sterilization. Optionally, moisture used to activate the sterilizing agent is sufficient to produce the desired relative humidity (RH) within the sterilization chamber. The system according to an aspect of the disclosed concept further employs a locking mechanism and control system to ensure that the self-contained sterilization chamber remains sealed until a predefined condition, as determined by the control system, is satisfied. In some embodiments, the control system includes a timer that directs the locking mechanism to transition from a locked configuration into an unlocked configuration after a desired amount of time has elapsed. Preferably, the locking mechanism is a fail-deadly device that, once tripped, remains engaged until the control system determines that the predefined condition is satisfied. Accordingly, the locking mechanism prevents the self-contained sterilization chamber from being accessed until the predefined condition is met. Further, the locking mechanism prevents the sterilization chamber from being accessed even after a loss of power or other catastrophic failure. Thus, the disclosed concept provides a sterilization system that prevents the unwanted dispersal of the sterilizing agent from the self-contained sterilization chamber into the ambient environment.

To achieve the above-described functionalities, in one aspect, the disclosed concept comprises an isolation unit 1 having at least one hatch 2, at least one locking mechanism 3, at least one HMI device 4, at least one output device 5 and a control system 6. The isolation unit 1 is an enclosure used to perform sterilization operations and the hatch 2 is integrated into the isolation unit 1 so that a user is able to access an interior compartment 13 of the isolation unit 1 through the hatch 2. The locking mechanism 3 is operatively coupled in between the isolation unit 1 and the hatch 2. This coupling enables the locking mechanism 3 to function as a fail-deadly switch to prevent opening the hatch 2 once the locking mechanism 3 is engaged. Specifically, the locking mechanism 3 is designed to prevent the interior compartment 13 from being accessed until a predefined condition is satisfied. Thus, the user is prevented from opening the hatch 2 while the locking mechanism 3 is engaged. Additionally, the user is given no means of disengaging the locking mechanism 3, once engaged. This functionality limits the potential for the user to be exposed to harmful sterilizing agents.

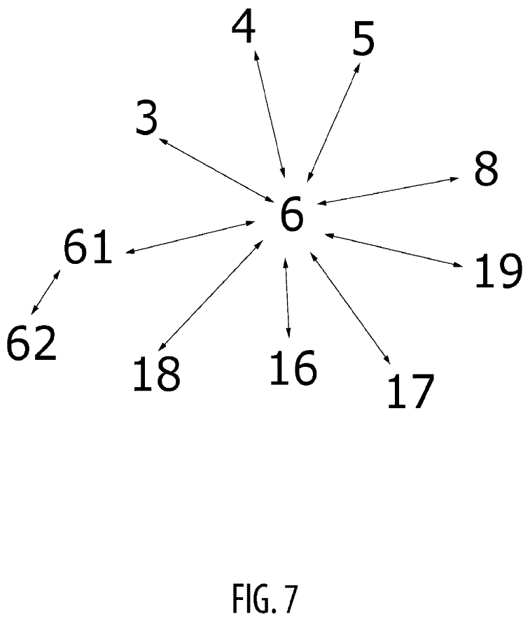

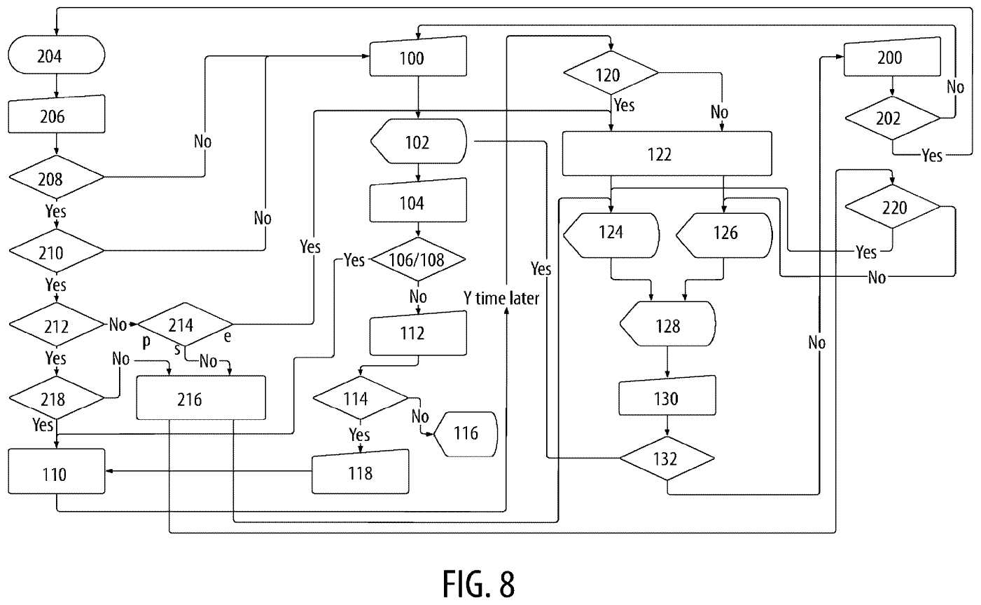

Referring to FIG. 7 the disclosed concept is designed to provide an automated system for determining when to disengage the locking mechanism 3. Accordingly, the control system 6 is designed to operate as a central processing hub for commands and data and can be any computing device used to direct the operation of the electrical components of the disclosed concept (e.g., a microcontroller, an integrated circuit, a personal computing device, a smartphone, or a tablet computer). Additionally, the control system 6 is designed to provide the control signals required to disengage the locking mechanism 3 once the predefined conditions are satisfied. That is, the locking mechanism 3 will remain locked until the disengagement signal is received from the control system 6. Further, even power loss will not force the locking mechanism 3 to be disengaged. In some embodiments, an external system 8 (e.g., a remote server, a personal computing device, a smartphone, or a tablet computer) may be communicably coupled to the control system 6 such that the external system 8 may monitor and control the isolation unit 1. The disclosed concept optionally makes use of the HMI device 4 to enable the user to initiate a sterilization operation and to relay commands to the control system 6 (FIG. 7). The HMI device 4 is preferably at least one device selected from the group consisting of buttons, keylock switches, dials, keypads, switches, touch screens and human input devices. The output device 5 is designed to relay information to the user. Preferably, the output device 5 is at least one member selected from the group consisting of speakers, display devices, lamps, indicator lights, vibration motors, heating elements, and touch screens. The HMI device 4 and the output device 5 are optionally laterally mounted onto the isolation unit 1. Consequently, the HMI device 4 and the output device 5 are advantageously positioned to relay information between the user and the isolation unit 1. The control system 6 is disposed within the isolation unit 1 and is communicably coupled to the HMI, the locking device, and the output device 5. As a result, the control system 6 is isolated from hazards in the external environment and is able to govern the operation of all electrical components of the disclosed concept. Further, the control system 6 is able to generate commands that direct the locking mechanism 3 to become engaged and then disengaged. In some embodiments, the control system 6 is communicably coupled to a timer 61 (FIG. 7) to determine when to disengage the locking mechanism 3. The timer 61 is designed to track the amount of time that has elapsed between relevant events of the sterilization operation. Optionally, the timer 61 may include a self-contained power supply 62 (e.g., a battery, generator, or fuel cell) so that the timer 61 continues to function regardless of power being supplied to the remaining components of the isolation unit 1. In some embodiments, the timer 61 includes an internal counter to track the number of power interruptions and power cycles that occur within a given time period (FIG. 8).

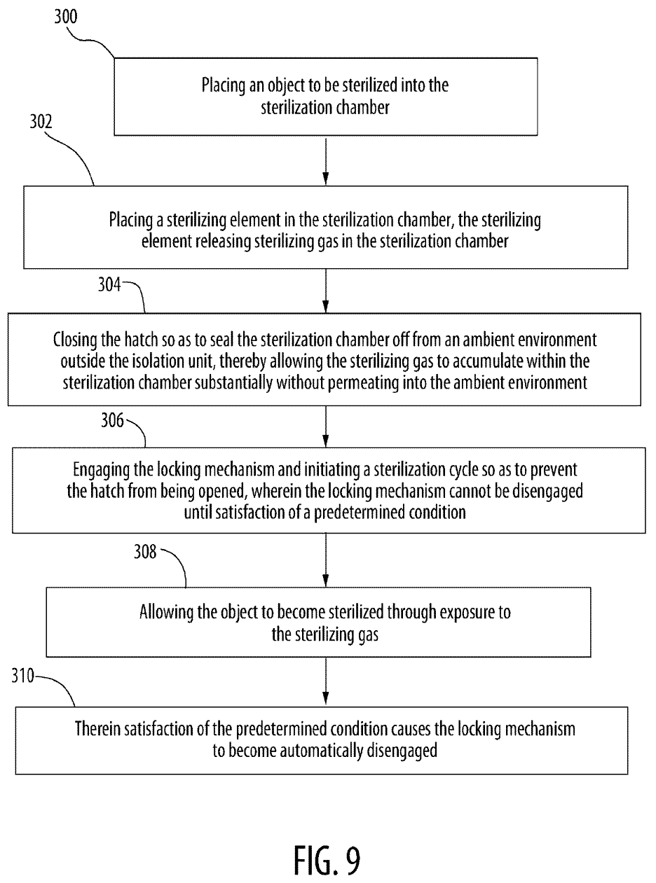

As seen in FIG. 9, an optional method for employing the system of the disclosed concept to perform a sterilization operation is as follows. The method begins by loading an object(s) to be sterilized into the interior compartment 13 of the isolation unit 1. A sterilizing element 7 is then placed into the interior compartment 13. In some embodiments, the sterilizing element is optionally a three-phase entrained polymer component comprising a base polymer, chlorine dioxide releasing agent and channeling agent, as described herein. The sterilizing element 7 is optionally activated through contact with water either immediately before or after placing the sterilizing element 7 into the interior compartment 13. Optionally, an automatic activation mechanism is disposed within the interior compartment 13 and is used to dispense a desired amount of sterilizing element 7 into the interior compartment 13 and then add a required amount of fluid to sustain the chemical reaction. The user then closes the hatch 2 and engages the locking mechanism 3. The sterilizing element 7 begins to release the sterilizing agent into the interior compartment 13. Preferably, the sterilizing agent is a gas, preferably chlorine dioxide gas, that sterilizes the object(s) within the interior compartment 13 before breaking down into benign compounds. The control system 6 then tracks the time with the timer 61 to determine when it is safe to automatically disengage the locking mechanism 3. Once the control system 6 determines that it is safe to open the hatch 2, a command is sent to the locking mechanism 3 to disengage. The user is then able to remove the sterilized object(s) from the interior compartment 13 by opening the hatch 2. Optionally, the timer 61 generates the disengagement command if a predetermined amount of time has passed.

In some embodiments, the isolation unit 1 comprises a sterilization chamber 11 and a component enclosure 12, as shown in FIG. 1. The sterilization chamber 11 functions as the housing for sterilization operations. The hatch 2 is integrated into the sterilization chamber 11. Accordingly, the user is able to access the interior compartment 13 of the sterilization chamber 11 through the hatch 2. The component enclosure 12 is a housing for the electrical components of the disclosed concept. Optionally, the component enclosure 12 functions as the primary control panel for the isolation unit 1. Further, the component enclosure 12 may be laterally mounted onto the sterilization chamber 11. Thus positioned, the component enclosure 12 does not hinder opening or closing the hatch 2.

In some embodiments of the disclosed concept, the HMI device 4 and the output device 5 are mounted onto the component enclosure 12 such that an exterior surface of the component enclosure 12 acts as a control panel. Specifically, in some embodiments, the HMI device 4 comprises at least one control button 41, and at least one keylock switch 42 (FIG. 1). The control button 41 enables the user to enter commands into the isolation unit and preferably enables the user to start the timer 61 for the locking mechanism 3 to lock the hatch 2. The keylock switch 42 enables the user to manually reset the number of sterilization operation cycles that the disclosed concept has recorded. Additionally, in some embodiments, the output device 5 comprises at least one status light 51, at least one primary display device 52, and at least one secondary display device 53. In further embodiments, the at least one status light 51 is a plurality of status lights that includes a maintenance alert light and a system status light.

In some embodiments, the component enclosure 12 is a shell or casing that is mounted around the sterilization chamber 11 such that the sterilization chamber 11 is housed within the component enclosure 12. In these embodiments, the hatch 2 is optionally integrated into the component enclosure 12 and the sterilization chamber 11 such that access to the interior compartment 13 is governed by opening and closing the hatch 2. Optionally, the hatch 2 is dorsally disposed on the isolation unit 1. The component enclosure 12 optionally includes at least one cooling fan that is disposed within the component enclosure 12 and communicably coupled to the control system 6. Additionally, at least one ventrally disposed intake vent, and at least one dorsally disposed exhaust vent are optionally integrated into the component enclosure 12 so that the cooling fan is able to move air through the component enclosure 12 and maintain the electronic components of the isolation unit 1 at desired temperatures. In an optional embodiment, an environmental sensor probe is disposed adjacent to the intake vent such that the environmental sensor probe is exposed to unadulterated ambient air. The environmental sensor probe is communicably coupled to the control system 6 so that the user is alerted whenever ambient conditions (e.g., temperature and relative humidity) are not within a predefined range. Optionally, the user is prevented from initiating a sterilization operation if ambient conditions are not within the predefined range. Optionally, the environmental sensor probe functions as an environmental monitoring system (EMS) that monitors the ambient environment to detect any leaks of sterilizing gas from the sterilization chamber 11 and alert the user if a hazardous concentration of sterilizing gas is detected.

The sterilizing agent may be caustic or corrode the materials of the sterilization chamber 11 and the hatch 2, so some components may need to be replaced periodically. For example, if the sterilizing agent is chlorine dioxide gas, the gas may degrade an elastomeric gasket that helps to seal the hatch 2. The gasket may therefore need to be replaced after a certain number of sterilization operation cycles. To facilitate timely replacements, the control system 6 tracks the number of sterilization operation cycles the system performs. The control system 6 then directs the secondary display device 53 (FIG. 1) to visually output the number of sterilization operation cycles that have occurred since the last time the user actuated the keylock switch 42 to reset the control system's 6 cycle counter. If the number of cycles exceeds a maintenance threshold, the control system 6, optionally, directs the maintenance alert light to turn on. The primary display device 52 is, optionally, used to visually output the amount of time remaining until the locking mechanism 3 is disengaged. In these embodiments, the control button 41, the keylock switch 42, the status light 51, the primary display device 52, and the secondary display device 53 are all laterally mounted onto the component enclosure 12 and serve to form a control panel for relaying information between the user and the isolation unit 1.

The hatch 2 comprises a door 22 and an opening 21. The opening 21 traverses through the sterilization chamber 11 into the interior compartment 13 and the door 22 is mounted over the opening 21. Consequently, the door 22 is able to prevent or allow access into the interior compartment 13. In some embodiments, as noted above, a gasket is mounted around the opening 21 to ensure that the door 22 hermetically seals the interior compartment 13 once closed. In some embodiments, the sterilization chamber 11 and the hatch 2 are thermally insulated. Optionally, the volume of the interior compartment 13 ranges from 64 in³ to 20,736 in³. In one embodiment, the volume of the interior compartment 13 is 3888 in³.

The locking mechanism 3 is designed to resist becoming disengaged once deployed. To facilitate this, in some embodiments of the disclosed concept the locking mechanism 3 comprises an engagement device 31 and a receptacle 35. The engagement device 31 is a male-pronged system that engages into the receptacle 35 to lock the hatch 2. The engagement device 31 is mounted onto the hatch 2 and the receptacle 35 is mounted onto the sterilization chamber 11. More specifically, the engagement device 31 optionally comprises at least one locking prong 32, at least one biasing device 33, at least one detent 36, and at least one reset lever 34. The biasing device 33 is an actuator that continually applies a force to the locking prong 32 that drives the locking prong 32 toward and into the receptacle 35. The detent 36 is operatively coupled in between the locking prong 32 and the biasing device 33 such that the detent 36 prevents the locking prong 32 from being displaced until released. In some embodiments, the detent 36 is released under a command from the control system 6. Once the detent 36 is released, the locking prong 32 is engaged into the receptacle 35 to lock the hatch 2 (FIG. 1). The biasing device 33 then provides the requisite force to maintain the locking prong 32 in this locked configuration. The biasing device 33 may be any component selected from the group consisting of springs, pneumatic cylinders, linear actuators, and rotary actuators, for example. In further embodiments, the detent 36 is reengaged once the locking prong 32 is deployed to ensure the locking prong 32 remains engaged into the receptacle 35 until the control system 6 issues the appropriate command. Further, this engagement may be designed to remain in place regardless of power being supplied to the isolation unit 1. The reset lever 34 protrudes from the locking prong 32 and is used to disengage the locking prong 32 from the receptacle 35. Thus, the reset lever 34 is able to move the locking mechanism 3 into the unlocked configuration and to reset the detent 36 to retain the locking prong 32 in a charged state. In some embodiments, a reset actuator is used to disengage the locking prong 32 from the receptacle 35. In some embodiments, the reset lever 34 is prevented from disengaging the locking prong 32 without first receiving instructions from the control system 6 or without satisfaction of another predefined condition (e.g., a time window expiring). A portable power supply (e.g., solar cells, a generator, a rechargeable battery) is optionally coupled to the isolation unit 1 to enable the system to function independent of an external power supply.

In alternative embodiments, the locking mechanism 3 comprises a magnetic lock. Further, alternative mechanical locking mechanisms 3 are contemplated as being within the scope of a locking mechanism 3 as that term is used herein. In additional alternative embodiments, the user has no interaction with the locking mechanism 3. That is, the locking mechanism 3 is integrated in between the hatch 2 and the sterilization chamber 11 such that the user does not have to actuate the release lever 34 to open the hatch 2. Rather, the locking mechanism 3 relies on commands from the control system 6 to become engaged or disengaged. Further, a handle is optionally mounted onto the hatch 2 to facilitate opening and closing the hatch 2 once the locking mechanism 3 has been disengaged.

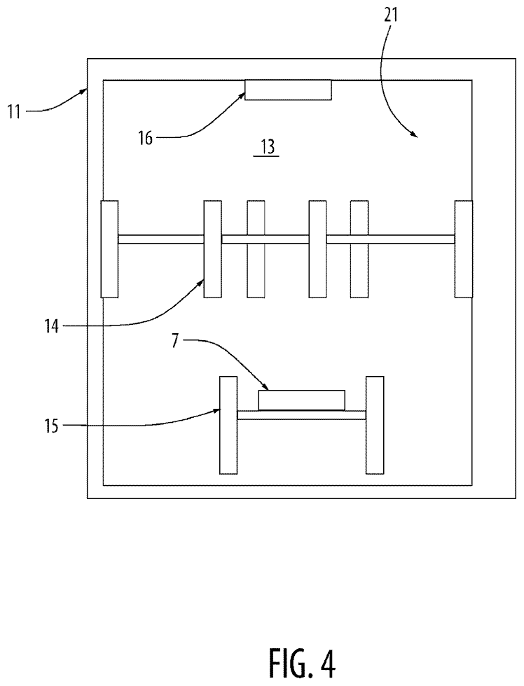

As seen in FIG. 4, the disclosed concept is designed to facilitate performing safe and efficient sterilization operations. To facilitate this, embodiments of the disclosed concept optionally comprise an environmental analysis instrumentation suite 16, a rack assembly 14, and an element tray 15. The environmental analysis instrumentation suite 16 is a collection of manipulators and measuring tools designed to monitor the environment within the interior compartment 13. In supplemental embodiments, the control system 6 uses additional sensor data to determine if the predefined conditions are satisfied before disengaging the locking mechanism 3. The rack assembly 14 is mounted within the interior compartment 13 and functions as a mounting system for objects within the interior compartment 13. The element tray 15 is mounted within the interior compartment 13 and serves as a platform for the sterilizing element 7 during sterilization operations.

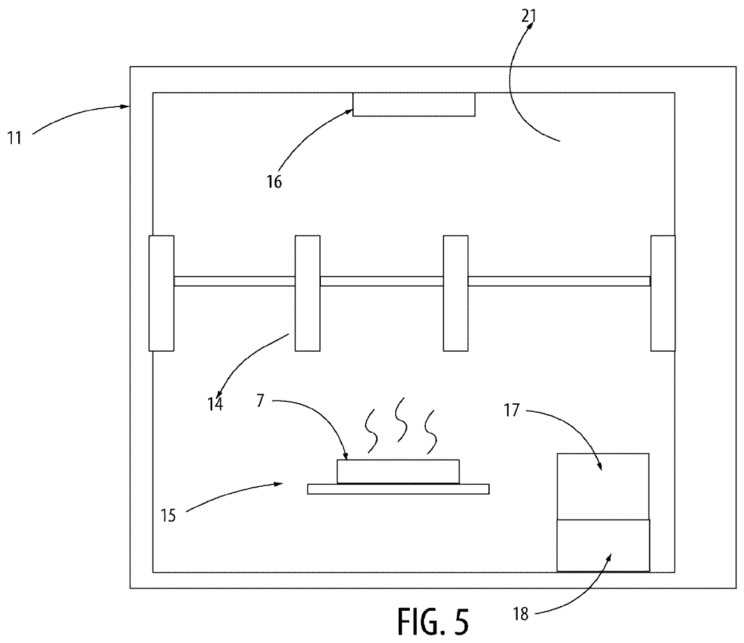

Referring to FIG. 5, the isolation unit 1 may further include an environmental control system that includes a temperature control system 17 thermally coupled to the interior compartment and a humidity control system 18 in fluid communication with the interior compartment 13. The temperature control system 17 may modify the temperature within the interior compartment 13 to facilitate completion of the sterilization operation and may increase or decrease the temperature to change the character of the chemical reactions occurring within the interior compartment 13. The humidity control system 18 may modify the relative RH within the interior compartment 13 to facilitate completion of the sterilization operation and may increase or decrease the RH to change the character of the chemical reactions occurring within the interior compartment 13. Both the temperature control system 17 and the humidity control system 18 are communicably coupled to the control system 6. In some exemplary non-limiting embodiments, the interior compartment 13 is maintained between 23 degrees and 60 degrees Celsius during the sterilization operation. In some exemplary non-limiting embodiments, the interior compartment 13 is maintained within as RH of 50% to 90% during the sterilization operation. In some exemplary non-limiting embodiments, the RH is limited to a range of 60% to 70%.

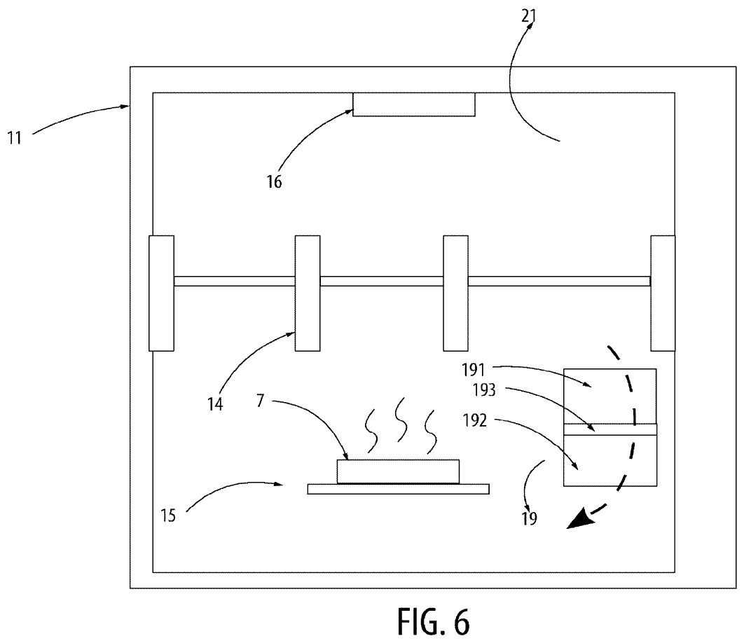

Referring to FIG. 6, the isolation unit may further comprise an air purification system 19 that is in fluid communication with the interior compartment 13 and is communicably coupled to the control system 6. Optionally, the air purification system 19 is a recirculation filtration system that neutralizes or removes contaminants within the interior compartment 13 without exhausting contaminants into the external ambient environment. To facilitate this, the air purification system may comprise a filtration element 191 and a pump 192. In some embodiments gases within the interior compartment 13 are pulled into the filtration element 191 and then exhausted back into the interior compartment by the pump once the desired amount of contaminants has been removed by the filtration element. Further, positioning the filtration element 191 between the interior compartment 13 and an inlet of the pump 192 ensures that the components of the pump 192 are not degraded or contaminated by contact with the unfiltered gases within the interior compartment 13. In some embodiments, the air purification system 19 is activated after the satisfaction of a secondary predetermined condition. The secondary predetermined condition refers to a secondary event that the control system 6 looks for as a trigger to activate the air purification system 19. Optionally, the secondary predetermined condition is an amount of time or a desired chemical composition within the headspace. The secondary predetermined condition is an event that happens in addition to the event that satisfies the original (primary) predetermined condition for disengaging the locking mechanism 3. Optionally, an emergency shutoff switch is communicably coupled to the air purification system 19 and the control system 6 so that the user may activate the air purification system 19 to neutralize the sterilizing element 7 and any sterilizing gas within the headspace. Optionally, the air purification system 19 is automatically activated to neutralize the sterilizing element 7 and any sterilizing gas within the headspace if the interior compartment 13 is breached before the predefined condition is satisfied. The filtration element 191 may further include an activated carbon component 193 disposed such that gas flowing through the filtration element passes through the activated carbon component 193. The activated carbon component 193 may be a removable cartridge, a refillable hopper, or a combination of the two. Optionally, an access door is integrated into the isolation unit 1 so that the activated carbon component 193 may be extracted or replaced. Optionally, the activated carbon component 193 contains between 5-25 g activated carbon.

The following exemplary embodiments further describe optional aspects of the presently disclosed technology with reference to FIG. 8, and are part of this Detailed Description. These exemplary embodiments are set forth in a format substantially akin to claims, although they are not technically claims of the present application. The following exemplary embodiments refer to each other in dependent relationships as “embodiments” instead of “claims.”
1. A method for sterilizing an object, the method comprising:
   prompting to open a hatch door of an isolation unit (100);
   placing an object to be sterilized into a sterilization chamber within the isolation unit (102);
   placing a sterilizing element in the sterilization chamber, the sterilizing element releasing sterilizing gas in the sterilization chamber (102);
   closing the hatch so as to seal the sterilization chamber off from an ambient environment outside the isolation unit, thereby allowing the sterilizing gas to accumulate within the sterilization chamber substantially without permeating into the ambient environment (104);
   prompting to begin a sanitization cycle and initializing an alarm countdown timer (106);
   determining if at least one cycle start condition is satisfied (108), wherein a cycle countdown timer is initialized if the at least one cycle start condition is satisfied (110), and wherein the hatch door is permitted to be opened if the cycle start condition is not satisfied (112);
   performing one or more of the following based on a state of the hatch door (114): (i) generating an alert if the hatch door is not closed, as described hereinabove, before the alarm countdown timer expires (116), (ii) prompting to begin the sanitization cycle if the hatch door is closed, as described hereinabove, and the alarm countdown timer expires (118), and (iii) executing 110 if the hatch door is closed, as described hereinabove, and the alarm countdown timer expires;
   determining if at least one reaction complete condition is satisfied (120);
   enabling post-processing procedures after the cycle countdown timer expires (122), wherein at least one primary post-processing procedure (124) is executed if the at least one reaction complete condition is satisfied, and wherein at least one secondary post-processing procedure (126) is executed if the at least one reaction complete condition is not satisfied;
   designating the sanitization cycle complete after the conclusion of one or more post-processing procedures including 124 and 126 (128);
   prompting to open the hatch door (130); and
   prompting to perform a subsequent sanitization cycle (132).
2. The method of embodiment 1, wherein 102 is executed whenever the prompt to perform a subsequent sanitization cycle is accepted.
3. The method of embodiment 1 comprising:
   closing the door if the prompt to perform a subsequent sanitization cycle is not accepted (200);
   monitoring a power switch (202), wherein 100 is executed when the power switch is not switched off, and wherein the locking mechanism is engaged so as to prevent the hatch from being opened when the power switch is switched off (204), and wherein the locking mechanism cannot be disengaged until satisfaction of a predetermined condition (206);
   determining if a power cycle occurred due to an unplanned interruption or a planned event (208), wherein 100 is executed if the power cycle did not occur due to an unplanned interruption;
   determining if the power cycle occurred before the cycle countdown timer for a previously attempted sanitization cycle has expired whenever it is determined that the power cycle occurred due to an unplanned interruption (210), wherein 100 is executed if the power cycle did not occur before the cycle countdown timer for the previously attempted sanitization cycle expired;
   determining if the power cycle occurred before the at least one reaction complete condition was satisfied for the previously attempted sanitization cycle whenever it is determined that the power cycle occurred before the cycle countdown timer for the previously attempted sanitization cycle has expired (212);
   executing 124 if the cycle countdown timer for the previously attempted sanitization cycle expired while the power was off (214);
   resuming the cycle countdown timer for the previously attempted sanitization cycle if the cycle countdown timer is not expired (216), wherein the previously attempted sanitization cycle is subsequently designated as the sanitization cycle and 124 is executed once the cycle countdown timer expires;
   determining if the power resumed before a reaction countdown timer expires (218), wherein 110 is executed if the power is resupplied before the reaction countdown timer expires, and wherein 216 is executed if the power was not resupplied before the reaction countdown timer expired; and
   determining if at least one reaction complete indicator is available, if the power was not resupplied before the reaction countdown timer expired (220), wherein 124 is executed if at least one reaction complete indicator became available during the power outage, and wherein 126 is executed if at least one reaction complete indicator did not become available during the power outage.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the disclosed concept contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. An isolation unit for sterilizing an object, the isolation unit comprising:
a housing having an interior compartment comprising a sterilization chamber;
at least one hatch;
at least one locking mechanism;
at least one human machine interface (HMI) device;
at least one output device; and
a control system;
the hatch being integrated into the isolation unit so as to provide selective access to the sterilization chamber;
the locking mechanism being operatively coupled to the hatch, wherein the locking mechanism functions as a fail-deadly switch to prevent opening of the hatch once the locking mechanism is engaged;
the HMI device being mounted onto the isolation unit;
the output device being mounted onto the isolation unit;
the control system being disposed within the isolation unit; and
the control system being communicably coupled to the HMI device and the output device, wherein the control system is configured to send a signal operative to disengage the locking mechanism only upon satisfaction of a predefined condition.

2. The isolation unit of claim 1 wherein:
the housing comprises a component enclosure;
the component enclosure is mounted onto the chamber; and
the component enclosure is positioned offset from the hatch.

3. The isolation unit of claim 2 wherein:
the HMI device comprises at least one control button; and
the control button is mounted onto the component enclosure.

4. The isolation unit of claim 2 wherein:
the HMI device comprises at least one keylock switch; and
the keylock switch is laterally mounted onto the component enclosure.

5. The isolation unit of claim 2, wherein:
the output device comprises at least one status light; and
the status light is mounted onto the component enclosure.

6. The isolation unit of claim 2, wherein:
the output device comprises at least one primary display device and/or at least one secondary display device; and
the primary display device and/or the secondary device is mounted onto the component enclosure.

7-11. (canceled)

12. The isolation unit of claim 1, wherein:
the locking mechanism comprises an engagement device and a receptacle;
the engagement device is mounted onto the hatch;
the receptacle is mounted onto the chamber; and
the engagement device is selectively engaged into the receptacle, wherein this engagement prevents the opening from being accessed through the door.

13. The isolation unit of claim 12 wherein:
the engagement device comprises at least one locking prong and at least one biasing device; and
the locking prong is operatively coupled to the biasing device, wherein the biasing device forces the locking prong to selectively engage into the receptacle.

14. The isolation unit of claim 12 wherein:
the engagement device comprises at least one reset lever; and
the reset lever is operatively coupled to the biasing device, wherein the reset lever forces the locking prong to disengage from the receptacle.

15. The isolation unit of claim 1 comprising:
an environmental analysis instrumentation suite,
wherein the environmental analysis instrumentation suite is mounted within the interior compartment of the sterilization chamber.

16. The isolation unit of claim 15, wherein the control system comprises a microcontroller and wherein the microcontroller is communicably coupled to the locking mechanism and the environmental analysis instrumentation suite.

17-18. (canceled)

19. The isolation unit of claim 1 comprising:
a sterilizing element, wherein the sterilizing element produces a chlorine dioxide gas, once activated.

20. (canceled)

21. The isolation unit of claim 1 comprising:
a temperature control system thermally coupled to the interior compartment; and
the temperature control system is communicably coupled to the control unit.

22. The isolation unit of claim 1 comprising:
a humidity control system which is in fluid communication with the interior compartment,
wherein the humidity control system is communicably coupled to the control unit.

23. The isolation unit of claim 1 comprising:
an air purification system which is in fluid communication with the interior compartment,
the air purification system being communicably coupled to the control unit,
wherein the air purification system includes a filtration element and a pump, wherein a quantity of gas within the interior compartment is pulled into the filtration element and then exhausted back into the interior compartment by the pump once a desired amount of contaminants has been removed by the filtration element.

24. (canceled)

25. The isolation unit of claim 23, wherein gas flowing through the filtration element passes through an activated carbon component.

26. (canceled)

27. The isolation unit of claim 23, wherein the filtration element is a recirculation filter.

28. The isolation unit of claim 1 comprising:
a timer comprising a self-contained power supply; and
the timer being communicably coupled to the control system.

29. A method for sterilizing an object, the method comprising:
providing an isolation unit for sterilizing an object, the isolation unit comprising:
a housing having an interior compartment comprising a sterilization chamber;
a hatch that is integrated into the isolation unit so as to provide selective access to the sterilization chamber; and
a locking mechanism operatively coupled to the hatch, wherein the locking mechanism functions as a fail-deadly switch to prevent opening of the hatch once the locking mechanism is engaged;
placing an object to be sterilized into the sterilization chamber;
placing a sterilizing element in the sterilization chamber, the sterilizing element releasing sterilizing gas in the sterilization chamber;
closing the hatch so as to seal the sterilization chamber off from an ambient environment outside the isolation unit, thereby allowing the sterilizing gas to accumulate within the sterilization chamber substantially without permeating into the ambient environment;
engaging the locking mechanism and initiating a sterilization cycle so as to prevent the hatch from being opened, wherein the locking mechanism cannot be disengaged until satisfaction of a predetermined condition; and
allowing the object to become sterilized through exposure to the sterilizing gas; wherein satisfaction of the predetermined condition causes the locking mechanism to become automatically disengaged, wherein the predetermined condition is an amount of time and wherein prior to the locking mechanism being automatically disengaged, the locking mechanism cannot be disengaged through a user-initiated override.

30. The method of claim 29, wherein the amount of time is tracked by a timer, and wherein in the event of a loss of power to the isolation unit, upon restoration of power to the isolation unit, the timer either continues where it left off at the time power was lost or the timer begins over again to track the amount of time anew, wherein the timer cannot be manually reset or overridden so as to disengage the locking mechanism.

31. The method of claim 29, wherein an air purification system neutralizes the sterilizing gas within a headspace after the satisfaction of a secondary predetermined condition, the headspace being formed of a portion of the interior compartment that is not occupied by the object.

32. The method of claim 31, wherein the secondary predetermined condition is an amount of time or a desired chemical composition within the headspace.

33. (canceled)

34. A system for sterilizing an object, the system comprising:
an isolation unit;
a sterilizing element;
a locking mechanism;
a timer;
placing the object in the sterilization chamber;
placing the sterilizing element in the sterilization chamber;
initializing a cycle countdown timer; and
sealing the sterilization chamber with the locking mechanism, wherein the locking mechanism cannot be disengaged until the cycle countdown timer expires.

35. (canceled)

36. (canceled)

37. The method of claim 29, the sterilizing gas being chlorine dioxide gas.

38. The method of claim 37, the sterilizing element including a polymer composition comprising a blend of a base polymer and a chlorine dioxide gas forming agent, the method comprising activating the sterilizing element to produce the chlorine dioxide gas.
